(19) United States
(12) Patent Application Publication
(10) Pub. No.: US 2004/0016008 A1
(43) Pub. Date: Jan. 22, 2004

(54) Hybrid transgenic mouse with accelerated onsent of Alzheimer type amyloid plaques in brain

(76) Inventors: William Stephen Brimijoin, Rochester, MN (US); Tina Rees, Saint Charles, MN (US); Steven G. Younkin, Jacksonville, FL (US); Hermona Soreq, Jerusalem (IL)

Correspondence Address: John P. White, Cooper & Dunham LLP, 1185 Avenue of the Americas, New York, NY, 10036, US

(21) Appl. No.: 10/337,142

(22) Filed: Jan. 6, 2003

Related U.S. Application Data
(60) Provisional application No. 60/346,693, filed on Jan. 7, 2002.

(51) Int. Cl. A01K 67/027

(52) U.S. Cl. 800/18

(57) ABSTRACT

The invention provides a transgenic rodent whose genome comprises a mutant hAPP transgene and an hAChE transgene. Methods and materials for making such transgenic rodents, and using them to identify agents that affect the progression of Alzheimer's disease are also provided.

### PRIORITY

This application claims the benefit of U.S. Provisional Application No. 60/346,693, filed Jan. 7, 2002, the contents of which are hereby incorporated by reference into this application.

### STATEMENT AS TO FEDERALLY SPONSORED RESEARCH

Funding for the work described herein was provided by the United States government, which may have certain rights in the invention.

### FIELD OF THE INVENTION

The invention relates to methods and materials involved in making and using transgenic rodents that have two transgenes.

### BACKGROUND OF THE INVENTION

Alzheimer's disease (AD) is a neurodegenerative progressive disorder and the predominant cause of dementia in people over 65 years of age. The prevalence of AD is estimated to be as high as 18.7% among those within 75-84 years of age and 47.2% among those 85 years of age or older, affecting a significant portion of the population in most countries of the world. Clinical symptoms of the disease typically begin with subtle short-tern memory problems. As the disease progresses, difficulty with memory, language, and orientation worsen to the point of interfering with the ability of the person to function independently. Other symptoms, which are variable, include myoclonus and seizures.

The pathology in AD is confined exclusively to the central nervous system (CNS). Pathologic changes in AD brain include extracellular plaques of beta-amyloid (Aβ) and other proteins, intracellular neurofibrillary tangles of abnormally phosphorylated tau protein, and loss of cholinergic neurons in the basal forebrain. The relatively specific cholinergic deficit has led to current modalities of therapy intended to increase residual function. The most promising symptomatic treatments to date are based on drugs that reduce breakdown of the cholinergic neurotransmitter, acetylcholine, by inhibiting its metabolic enzyme, acetylcholinesterase (AChE).

In addition to a role in modulating synaptic acetylcholine levels, AChE appears to have non-cholinergic functions [Small, D. H. et al., Neurochemistry International, 28:453-483 (1996); Soreq, H. and Seidman S., Nature Reviews Neuroscience 2:294-302 (2001)], some of which may involve protein-protein interaction rather than enzymatic catalysis [Darboux, I. et al., EMBO J. 15: 4835-4843 (1996)]. Thus, studies on neuronal development suggest that AChE promotes neurite outgrowth [Karpel, R. et al. J. Neurochem. 66:114-123 (1996); Koenigsberger, C. et al., J. Neurochem. 69:1389-1397 (1997)], possibly through adhesive interactions with the extracellular matrix [Sharma, K. V. et al., J. Neurosci. Res. 63:165-175 (2001)]. In AD, the consistent presence of AChE in the senile plaques [Geula, C. et al., Brain Res. 644:327-330 (1994)] has led to the hypothesis that this protein might bind Aβ and promote its deposition. Indeed, in vitro, AChE has been shown to promote the formation of insoluble fibrils containing both Aβ and ACHE, which are more toxic to cells than Aβ alone.

Crystal structure data have suggested a potential locus of interaction for Aβ on the AChE molecule [Bourne, Y. et al., J. Biol. Chem. 274:2963-2970 (1999)]. This locus is near AChE's peripheral site, on the external surface of the enzyme near the entrance to its catalytic gorge. Consistent with this structural inference, peripheral site-directed AChE ligands like propidium iodide and certain monoclonal antibodies have been found to prevent AChE from enhancing the formation of amyloid fibrils in vitro [Inestrosa, N. C. et al., Neuron 16:881-891 (1996); Reyes, A. E. et al., Biochem. Biophys. Res. Com. 232:652-655 (1997)]. While these observations are intriguing, their relevance to the pathogenesis of Alzheimer's disease has been largely predictive. An appraisal of the influence of AChE on amyloid deposition in the intact brain is not yet available.

Tremendous effort has been spent to develop an animal model that exhibits classic AD pathology (neurofibrillary tangles and amyloid plaques) together with memory loss, a criterion for use in engineering and testing new compounds. Among the numerous transgenic mouse models produced to date, however, none has met this-criterion.

Mouse models of AD have been extensively characterized by many approaches, including batteries of behavioral tests, with varied results [Arendash, G. W. et al., Brain Res. 891:42-53 (2001); Erb, C. et al., J. Neurochem. 77(2):638-646 (2001); Katzman, R., Neurology 43(1):13-20 (1993); Kawarabayashi, T. et al., J. Neurosci. 21(2):372-381 (2001); King, D. L. et al., Behav. Brain. Res. 103:145-162 (1999)]. One of the most widely used models is the transgenic Tg2576 mouse [Gravina, S. A. et al., J. Biol. Chem. 270(13):7013-7016 (1995)], which incorporates the Swedish mutation of human APP (APPswe: K670M/N671L) to drive high expression of both Aβ 1-40 and 1-42. These mice have been reported to have age-dependent behavioral deficits beginning at six months of age or shortly after [Gravina et al., id ibid.; Samuel, W. et al., Arch. Neurol. 51(8):772-778 (1994)]. Tg2576 mice also show an age-dependent increase of insoluble amyloid beta in brain, first detected at 6 months by ELISA and later manifesting as histologically evident amyloid plaques at 9-10 months of age [Hsiao, K. et al., Science 274: 99-102 (1996)]. Consistent with the “amyloid hypothesis”, this amyloid burden has shown a weak negative correlation (r²≦0.36) with measures of reference memory [Samuel et al., id ibid.].

AChE is not itself a significant risk factor for AD. For example, a transgenic mouse has been developed to express human (h)AChE only in the CNS [Beeri, R. et al., Curr. Biol. 5(9):1063-1071 (1995)]. This mouse exhibits an overall 2-fold increase in total brain AChE activity, but does not develop amyloid plaques or neurofibrillary tangles. However, neuronal AChE overexpression impairs the cholinergic homeostasis in the mammalian brain [Soreq and Seidman, id. ibid.].

Thus, while animal models for AD are being rapidly developed, none have been able to closely mimic the disease progression in human subjects. A primary criterion for diagnosis of AD in early stages is memory loss, especially of working memory [Baddeley AD, et al., Brain 114:2521-42 (1991)]. This memory loss is associated with cholinergic allostasis [Coyle, Science (1983)], and therefore, a good animal model of AD would be one with working memory impairment that correlates with both impaired cholinergic transmission and increased amyloid burden.

Working memory differs greatly from reference memory, a more common subject of investigation [Holcomb L A, et al., Behav Genet 29:177-85 (1999); Hsiao K, et al., Science 274:99-102 (1996)]. The standard Morris Water Maze is a reference memory task, requiring the mice to remember the location of a submerged platform from day to day. Working memory tasks, however, require the animal to learn anew each day the position of the platform and to remember it for the rest of the day. Impairment in such tasks more closely resembles the memory deficits of early AD patients [Baddeley et al., id ibid.]. Because working memory tasks are more difficult than reference memory tasks, they are more powerful in distinguishing between relatively small impairments in small groups of animals. In order to test the working memory of the novel transgenes which are one of the objects of the present invention, a version of the radial arm water maze was used. Although this test was developed by Diamond et al. [Diamond D M, et al., Hippocampus 9(5):542-52 (1999)], it is not known to had been used to evaluate cognitive performance in the Tg2576 line with the APPswe mutation.

It is an object of the present invention to generate an improved AD animal model, which would enable exploring the possibility that cholinergic allostasis, or AChE excess by its own merit. This would enhance the development of AD pathology in the presence of high levels of amyloid beta, the inventors have crossing the hAChE and APPswe expressing lines, yielding the doubly trans genic progeny of the invention, which indeed showed not only accelerated amyloid deposition as compared with singly transgenic APPswe littermates and Tg2576 mice, but also cognitive changes. This double transgenic animal model and it uses are major objects of the present invention.

### SUMMARY OF THE INVENTION

In a first aspect, the present invention relates to a transgenic rodent having a genome comprising a mutant APP transgene and an AChE transgene, which transgenic rodent displays an altered deposition of amyloid beta in the brain, compared to a corresponding transgenic rodent whose genome comprises a mutant APP transgene and lacks an AChE transgene and progeny thereof.

By “corresponding transgenic rodent” as used herein is meant a transgenic rodent of the same species, sex, and substantially the same age and weight, that has been raised and treated under substantially identical conditions.

In one preferred embodiment, the transgenic rodent of the invention may be a mouse.

According to a preferred embodiment, the transgenic rodent of the invention comprises a AChE transgene encoding a wild-type AChE polypeptide. Preferably, this wild-type AChE polypeptide may be a full-length human AChE polypeptide.

In another specifically preferred embodiment, the transgenic rodent of the invention further comprises a mutant APP transgene which encodes a human APP mutated polypeptide. More specifically, such APP mutated polypeptide may be selected from the group consisting of APP695, APP751, app563, app714 and APP770, and has a mutation selected from the group consisting of K670M, N671L, V717I, V717G, or V717F.

In a particularly preferred embodiment, the transgenic rodent of the invention has a genome comprising a K670M/N671L mutant APP transgene and full-length human AChE polypeptide transgene.

Still further, the transgenic rodent provided by the invention exhibits deposition of thioflavin-S reactive plaques in its brain, the number of plaques being statistically significantly increased compared to the number of plaques in a corresponding transgenic rodent whose genome has a mutant APP transgene and lacks an AChE transgene.

According to another preferred embodiment, the statistically significant increase in thioflavin-S reactive plaques can occur earlier in age compared to a corresponding transgenic rodent whose genome comprises a mutant APP transgene and lacks an AChE transgene. More specifically, such statistically significant increase can be observed at an age of from about 6 to about 9 months of age.

Still further, the level of SDS-extractable amyloid beta in the brain of the transgenic rodent, preferably mouse, of the invention may be altered compared to a corresponding transgenic rodent whose genome comprises a mutant APP transgene and lacks an AChE transgene and progeny thereof.

In another specifically preferred embodiment, the level of SDS-extractable amyloid beta of the transgenic rodent of the invention may be elevated compared to a corresponding transgenic rodent whose genome comprises a mutant APP transgene and lacks an AChE transgene and progeny thereof.

The invention further provides for a method for generating a transgenic rodent. This method comprises the step of crossing a first transgenic rodent with a second transgenic rodent, wherein the genome of the first transgenic rodent comprises a mutant APP transgene and the genome of the second transgenic rodent comprises an AChE transgene.

In another preferred embodiment, the method for preparing a transgenic rodent according to the invention comprises the step of crossing a transgenic rodent whose genome comprises both a mutant APP transgene and an AChE transgene with a rodent whose genome lacks the mutant APP transgene and the AChE transgene.

In yet another embodiment, the method of the invention further comprises the step of identifying at least one progeny of the cross whose genome comprises the mutant APP transgene and the AChE transgene.

Still further, such progeny may have an altered level of SDS-extractable amyloid beta compared to the level of SDS-extractable amyloid beta in a corresponding transgenic rodent whose genome comprises a mutant APP transgene and lacks an AChE transgene.

Preferably, the level of SDS-extractable amyloid beta in the progeny may be statistically significantly increased, compared to the level in a corresponding rodent whose genome comprises a mutant APP transgene and lacks an AChE transgene.

Moreover, the number of plaques found in brains of the progeny, may be altered compared to a corresponding transgenic rodent whose genome comprises a mutant APP transgene and lacks an AChE transgene, particularly the number of plaques is elevated.

The invention further relates to a method of identifying a transgenic rodent comprising selecting, among a plurality of rodents, at least one rodent whose genome comprises a mutant APP transgene and an AChE transgene. More preferably, such rodent may be a mouse.

In a particularly preferred embodiment, such mouse has an altered, particularly elevated level of SDS-extractable amyloid beta compared to the level of SDS-extractable amyloid beta in a corresponding transgenic mouse whose genome comprises a mutant APP transgene and lacks an AChE transgene and/or has an altered, particularly elevated number of plaques in its brain compared to a corresponding transgenic rodent whose genome comprises a mutant APP transgene and lacks an AChE transgene.

In a second aspect, the present invention relates to a method of screening for a candidate substance for the treatment of a neurodegenerative disorder, which screening method comprises the steps of: (a) providing a test transgenic rodent whose genome comprises a mutant APP transgene and an AChE transgene; (b) administering said test substance to said transgenic rodent under suitable conditions; and (c) determining the effect of the test substance on an end-point indication, wherein said effect is indicative of the potential therapeutic effect of said test substance on said neurodegenerative disorder; and optionally (d) comparing said end-point indication with that of a control transgenic rodent not treated with said test substance.

In one specifically preferred embodiment of said aspect, the screening method of the invention comprises the steps of: (a) providing a test transgenic rodent whose genome comprises a K670M/N671L mutant APP transgene and full-length human AChE polypeptide transgene; (b) administering said test substance to said transgenic rodent under suitable conditions; and (c) determining the effect of the test substance on an end-point indication, wherein said effect is indicative of the potential therapeutic effect of said test substance on said neurodegenerative disorder; and optionally (d) comparing said end-point indication with that of a control transgenic rodent not treated with said test substance.

The test transgenic rodent of the invention has a statistically significantly elevated level of SDS-extractable amyloid beta and an increased number of thioflavin-S reactive plaques compared to a corresponding transgenic rodent whose genome comprises a mutant APP transgene and lacks an AChE transgene.

According to a particularly preferred embodiment, the test transgenic rodent may be any of the transgenic rodents defined by the invention.

In one specific embodiment, the end-point used by the screening method of the invention may be the level of SDS-extractable amyloid beta obtained from a brain tissue of the transgenic rodent. Where such end-point is used, a decrease in the level of SDS-extractable amyloid beta obtained from the test transgenic rodent brain tissue compared to the level of SDS-extractable amyloid beta in a control transgenic rodent not treated with said test substance, may be indicative of a therapeutic effect of said substance on a neurodegenerative disorder.

According to an alternative embodiment, the end-point used by the screening method of the invention may be any one of the number, density and burden of thioflavin-S reactive plaques in the brain of said transgenic rodent as determined histologically. Decrease in the number, density or burden of thioflavin-S reactive plaques in the brain of the test transgenic rodent compared to the plaques in the brain of said control transgenic rodent not treated with said test substance, is indicative of a therapeutic effect of said substance on the neurodegenerative disorder.

Another alternative end-point used by the screening method of the invention, may be a cognitive capacity, particularly working memory, as evaluated by a behavioral assay. A preferred behavioral assay may be any one of Morris water maze, the radial arm water maze and open field anxiety and exploration test. Improvement of the cognitive capacity in the test transgenic rodent compared to the cognitive capacity of the control transgenic rodent not treated with said test substance is indicative of a therapeutic effect of said substance on a neurodegenerative disorder.

The pace of said improvement may be indicative of the drug efficacy.

In a specifically preferred embodiment, the neurodegenerative disorder may be any one of Alzheimer's disease, Parkinson's disease, Gertsmann-Strausseler-Scheinker Syndrom, Fatal Familial Insomnia, Huntington's Chorea and Familial amiloid polyneropathy, which are generally associated with formation of amyloid plaques in the brain. Most particularly, such disorder is Alzheimer's disease.

Still further, the test substance tested by the screening method of the invention may be any substance selected from the group consisting of: protein based, carbohydrate based, lipid based, nucleic acid based, natural organic based, synthetically derived organic based, metals and antibody based substances.

According to another embodiment, the protein, nucleic acid, chemical or antibody based substance may be a product of a combinatorial library.

The present invention further provides a method of making a therapeutic composition for the treatment of a neurodegenerative disorder, which method comprises the steps of: identifying a substance having a therapeutic effect on a neurodegenerative disorder by the screening method defined by the invention and admixing said inhibitor with a pharmaceutically acceptable carrier.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety, including references cited therein. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 Bar graph depicting the levels of SDS-extractable amyloid beta detected with antibodies recognizing amyloid beta 1-40 (SDS 1-40), SDS-extractable amyloid beta detected with antibodies recognizing amyloid beta 1-42 (SDS 1-42), formic acid-extractable amyloid beta detected with antibodies recognizing amyloid beta 1-40 (FA 1-40), and formic acid-extractable amyloid beta detected with antibodies recognizing amyloid beta 1-42 (FA 1-42) in control, mutant hAPP (Tg2576), and hAPP/hAChE mice at 3 months of age. Values are expressed as mean pmol/g±standard error of the mean (SEM).

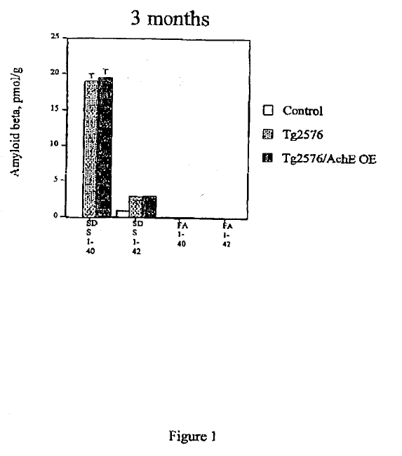

FIG. 2 Bar graph depicting the levels of SDS-extractable amyloid beta detected with antibodies recognizing amyloid beta 1-40 (SDS 1-40), SDS-extractable amyloid beta detected with antibodies recognizing amyloid beta 1-42 (SDS 1-42), formic acid-extractable amyloid beta detected with antibodies recognizing amyloid beta 1-40 (FA 1-40), and formic acid-extractable amyloid beta detected with antibodies recognizing amyloid beta 1-42 (FA 0.1-42) in control, mutant hAPP (Tg2576), and mutant hAPP/hAChE mice at 6 months of age. Values are expressed as mean pmol/g+SEM.

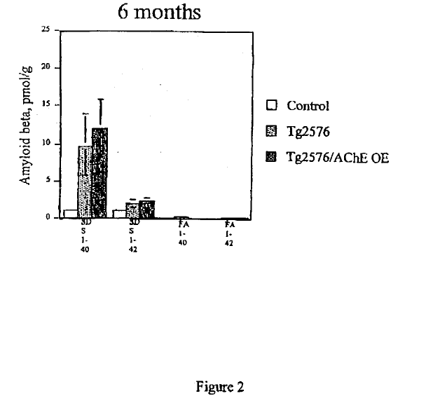

FIG. 3 Bar graph depicting the levels of SDS-extractable amyloid beta detected with antibodies recognizing amyloid beta 1-40 (SDS 1-40), SDS-extractable amyloid beta detected with antibodies recognizing amyloid beta 1-42 (SDS 1-42), formic acid-extractable amyloid beta detected with antibodies recognizing amyloid beta 1-40 (FA 1-40), and formic acid-extractable amyloid beta detected with antibodies recognizing amyloid beta 1-42 (FA 1-42) in control, mutant hAPP (Tg2576), and mutant hAPP/hAChE mice at 9 months of age. Values are expressed as mean pmol/g+SEM.

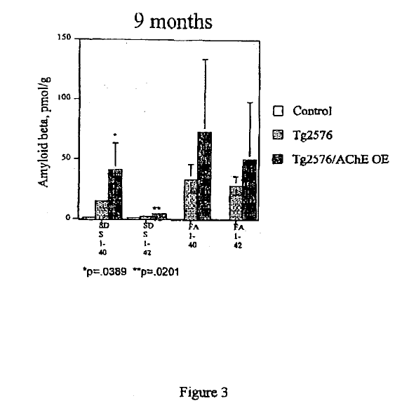

FIG. 4 Comparison of Thioflavin S-reactive plaques in hAPP and hAPP/hAChE cortex. (A) 6 month hAPP; (B) 6 month hAPP/hAChE; (C) 9 month hAPP; (D) 9 month hAPP/hAChE; (E) 12 month hAPP; (F) 12 month hAPP/hAChE. Scale bar represents 500 μm.

FIG. 5 Amyloid beta immunohistochemistry. Section of six month hAPP/hAChE cortex stained first with Thioflavin S (A), then stripped and restained for Aβ 1-42 (B). Six month hAPP/hAChE cortex stained first with Thioflavin S (C), then stripped and restained for Aβ 1-40 (D). Scale bar represents 100 μm.

FIG. 6 Immune components in amyloid plaques. Six month hAPP/hAChE cortex labeled for Thioflavin S (A) and stained for Cd11b (B). Six month hAPP/hAChE cortex double labeled for Thioflavin S (C) and GFAβ (D). Scale bar represents 100 μm.

FIG. 7 AChE immunoreactivity in amyloid plaques. (A) 12 month hAPP/hAChE cortex labeled with Thioflavin S. (B) Adjacent section (resulting in slight change of plaque topology) labeled for AChE by human-specific mouse monoclonal antibody.

FIG. 8 Schematic representation of radial arm water maze. Mice were swum for five one-minute trials per day to learn the position of a hidden platform. Errors were. counted to evaluate memory. These tracings are typical swim paths of mice during the task.

FIG. 9 Bar graph of radial arm water maze error data. Errors made per one-minute trial were averaged on trials 1 and 5 of each day. The improvement between trial 1 and trial 5 represents working memory acquisition. Data represents means±SEM. ANOVA p=0.0001, PLSD versus control *p<0.001.

FIG. 10 Representative Thioflavin S stained cerebral cortex of mice that underwent the water maze test. 12 μm sections of 9 month old mice were stained with 1% Thioflavin S to identify fibrillar amyloid. A. APPswe. B. APPswe/hAChE. Neither the control nor the singly transgenic hAChE mouse showed plaques (data not shown). Scale bar represents 100 μm.

FIG. 11 Quantitation of histologically identified amyloid of mice that underwent the water maze test. Brain sections from nine month mice were stained with 1% Thioflavin S and analyzed via NIH Image. Individual data points are shown. Group mean values are represented with horizontal black bars. A. Plaque density. B. Plaque burden. C. Average plaque area. Statistical significance of APPswe/hAChE versus APPswe determined by Mann-Whitney test. *p<0.05.

FIG. 12 Aβ ELISA of mice that underwent the water maze test. Insoluble amyloid beta 1-40 and 1-42 were determined from whole hemisphere homogenates in both genotypes at nine months. All data points are shown, and bars represent the mean values. Statistical significance of APPswe/hAChE versus APPswe determined by Mann-Whitney. *p=0.01.

FIG. 13 shows correlations of amyloid beta 1-42, plaque burden, and working memory. Histologically determined plaque burden correlated with (A, B) insoluble Aβ 1-42 and (C, D) total errors made in the radial arm water maze. A,C: APPswe/hAChE. B,D: APPswe. P values from a correlation Z-test are shown on each graph.

### DETAILED DESCRIPTION

The invention provides methods and materials related to a transgenic rodent whose genome contains a transgene encoding a mutant amyloid precursor protein (APP) and a transgene encoding acetylcholinesterase (AChE). A transgenic rodent expressing both transgenes exhibits properties useful in the study of Alzheimer's disease, including an altered level of SDS-extractable amyloid beta, an increase in the number, density and burden of plaques in the brain, and plaques that are detectable at an earlier age compared to the age of detectability in transgenic rodents carrying only the APP transgene. Transgenic rodents with such properties can be used to better understand Alzheimer's disease progression, or can be used to test whether or not a particular agent is capable of altering that progression and/or the toxicity and bioavailability of such agent.

Doubly Transgenic Non-Human Mammals

A doubly transgenic non-human mammal of the invention has a genome that comprises two transgenes: a mutant APP transgene and an AChE transgene. Such doubly transgenic non-human mammals are referred to herein as mutant APP/AChE transgenic non-human mammals. Transgenic non-human mammals are rodents such as rats, guinea pigs, hamsters and mice, and non-human primates such as baboons, monkeys, and chimpanzees.

A transgene useful in the invention encodes a polypeptide. Transgenes of the invention can be in a variety of forms. A transgene can, for example, consist of a wild type, full-length open reading frame (e.g., a cDNA). Such a transgene encodes a corresponding wild type, full-length polypeptide. Alternatively, a transgene can include introns or adjacent 5′- or 3′-untranslated regions (e.g., a genomic nucleic acid). In this case, the transgene can encode several different corresponding polypeptides determined in whole or in part by the interaction of genomic nucleic acid molecule components (e.g., exons, introns, or untranslated regions) with transcription or translation enzymes.

A transgene can be modified. Such modifications include, without limitation, additions, deletions, substitutions, point mutations, and combinations thereof. For example, a transgene can include a point mutation that enhances cleavage of the expressed polypeptide. Specific modifications such as point mutations can be introduced into the transgene by, for example, oligonucleotide-directed mutagenesis. In this method, a desired change is incorporated into an oligonucleotide, which then is hybridized to the transgene. The oligonucleotide is extended with a DNA polymerase, creating a heteroduplex that contains a mismatch at the introduced point change, and a single-stranded nick at the 5′ end, which is sealed by a DNA ligase. The mismatch is repaired upon transformation of E. coli, and the modified transgene encoding the corresponding modified polypeptide can be re-isolated from E. coli. Kits for introducing site-directed mutations can be purchased commercially. For example, Muta-Gene® in-vitro mutagenesis kits can be purchased from Bio-Rad Laboratories, Inc. (Hercules, Calif.).

PCR techniques also can be used to modify a transgene [see, for example, Vallette et al., Nucleic Acids Res. 17(2):723-733 (1989)]. PCR refers to a procedure or technique in which target nucleic acids are amplified. Sequence information from the ends of the region of interest or beyond typically is employed to design oligonucleotide primers that are identical in sequence to opposite strands of the template to be amplified, whereas for introduction of modifications, oligonucleotides that incorporate the desired change (e.g., addition, deletion, substitution, or mutation) are used to amplify the polynucleotide. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described in the literature [for example, in PCR Primer: A Laboratory Manual, Ed. by Dieffenbach, C. and Dveksler, G., Cold Spring Harbor Laboratory Press, 1995].

Transgenes also can be produced by chemical synthesis, either as a single polynucleotide molecule or as a series of polynucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a double-stranded nucleic acid molecule per:oligonucleotide pair, which then can be ligated into a vector.

In addition to a polynucleotide, a transgene also can include regulatory elements operably linked to the polynucleotide. Such regulatory elements may include promoter sequences, enhancer sequences, response elements, signal peptides, internal ribosome entry sequences, polyadenylation signals, terminators, or inducible elements that modulate expression (e.g., transcription or translation) of a polynucleotide. The choice of element(s) that may be included in a transgene depends upon several factors, including, without limitation, replication efficiency, selectability, inducibility, targeting, the level of expression desired, ease of recovery and the ability of the host to perform post-translational modifications. For example, a promoter can be included in a transgene to facilitate transcription of a polynucleotide. A promoter can be constitutive or inducible, and can affect the expression of a polynucleotide in a general or tissue-specific manner. Tissue-specific promoters include, without limitation, enolase promoter, prion protein (PrP) promoter, and tyrosine hydroxylase promoter. For example, a neuronal-specific enolase promoter can be included in a transgene to affect expression of a polynucleotide in neuronal tissues. Other promoters include, without limitation, platelet-derived growth factor (PDGF) promoter, simian virus 40 (SV40) promoter, and gene-specific promoters such as AChE or APP promoters. Regulatory elements can be from species including, without limitation, farm animals such as pigs, goats, sheep, cows, horses, and rabbits, rodents such as rats, guinea pigs, hamsters, and mice, non-human primates such as baboons, monkeys, and chimpanzees, or humans.

As used herein, “operably linked” refers to positioning of a regulatory element in a construct relative to a polynucleotide in such a way as to permit or facilitate expression of the encoded polypeptide. For example, a transgene can contain a neuronal-specific enolase promoter and a polynucleotide. In this case, the enolase promoter is operably linked to the polynucleotide such that it drives transcription of that polynucleotide in neuronal tissues.

Mutant APP Transgenes

Amyloid precursor protein (APP) polynucleotides for use in constructing an APP transgene are known in the art. APP polynucleotides are referred to by indicating the number of amino acids present in the corresponding expressed polypeptide. For example, APP770 refers to an APP polynucleotide that encodes an APP polypeptide with 770 amino acids. Other non-limiting examples of APP polynucleotides include APP563, APP695, APP714, and APP751. An APP polynucleotide can be from, for example, baboon, monkey, chimpanzee, or human (hAPP). As discussed herein, an APP polynucleotide useful in the invention is modified from its corresponding wild-type counterpart. Modifications to an APP polynucleotide that increase APP cleavage resulting in an increase in amyloid beta (Aθ) levels are particularly useful. For example, a hAPP polynucleotide containing K670M/N671L (i.e., the Swedish mutation), described in U.S. Pat. Nos. 5,877,399, 5,455,169, 5,850,003, all of which are incorporated by reference herein, can be used to construct an APP transgene of the invention. Expression of this mutant hAPP polynucleotide results in an increase in Aθ levels when the polynucleotide is expressed in the brain of a transgenic non-human mammal. Other suitable APP mutations can include, without limitation, V717I (i.e., the London mutation), V717G, and V717F (i.e., the Indiana mutation). Additional suitable transgenes encoding mutant APP polypeptides are discussed in U.S. Pat. No. 6,300,540, and also can be identified using methods known in the art, such as a search of the GenBank database.

AChE Transgenes

Acetylcholinesterase (AChE) polynucleotides for use in constructing an AChE transgene encode AChE polypeptides expressed in the brain. It is contemplated that a suitable AChE polynucleotide encodes an AChE polypeptide fragment containing one or more Aθ interaction sites. Typically, AChE polynucleotides are from human (hAChE). Additional suitable AChE transgenes are discussed in U.S. Pat. Nos. 5,932,780 and 6,258,780.

Generating Doubly Transgenic Non-Human Mammals

Various techniques can be used to generate doubly transgenic non-human mammals. Such techniques typically involve generating a plurality of non-human mammals whose genomes can be screened for the presence or absence of transgenes. Techniques for generating a plurality of non-human mammals include, without limitation, transforming cells with one nucleic acid construct containing two transgenes, transforming cells with two nucleic acid constructs each containing a single transgene, crossing two singly transgenic heterozygous mammals, crossing two singly transgenic homozygous mammals, and crossing doubly transgenic heterozygous or homozygous mammals from founder lines with non-transgenic mammals. For example, a first transgenic mouse heterozygous for a mutant hAPP transgene can be mated to a second transgenic mouse heterozygous for a wild type hAChE transgene and progeny identified that contain both the mutant HAPP transgene and the wild type hAChE transgene. In one embodiment, a male mouse overexpressing mutant hAPP (e.g., a Tg2576 mouse) is mated with female mice (e.g., a HpAChE mouse) overexpressing hAChE. Tg2576 and HpAChE mice are described in U.S. Pat. Nos. 5,877,399 and 5,932,780, respectively.

Various techniques can be used to introduce transgenes into a non-human mammal. Techniques to produce founder lines include, but are not limited to, pronuclear microinjection (U.S. Pat. No. 4,873,191), retrovirus mediated gene transfer into germ lines [Van der Putten et al., Proc. Natl. Acad. Sci. USA 82:6148 (1985)], gene targeting into embryonic stem cells [Thompson et al., Cell 56:313 (1989)], electroporation of embryos [Lo, Mol. Cell. Biol. 3:1803 (1983)], and transformation of somatic cells in vitro followed by nuclear transplantation [Wilmut et al., Nature 385(6619):810-813 (1997); Wakayama et al., Nature 394:369-374 (1998)]. When using mice to make a transgenic animal, suitable genetic backgrounds for use in making founder lines include, without limitation, C57B6, SJL/J, FVB/N, 129SV, BALB/C, C3H, and hybrids thereof.

Genotype

A doubly transgenic non-human mammal of the invention can be heterozygous for both transgenes, homozygous for both transgenes, or heterozygous for one transgene and homozygous for the other transgene. For example, a doubly transgenic mouse can be heterozygous for a mutant hAPP having the Swedish mutation and heterozygous for a wild-type hAChE transgene.

Initial screening to determine whether or not a genome comprises both transgenes can be accomplished by Southern blot analysis or PCR techniques [see, for example, sections 9.37-9.52 of Sambrook et al., 1989, “Molecular Cloning, A Laboratory Manual”, second edition, Cold Spring Harbor Press, Plainview, N.Y., for a description of Southern analysis]. Further, expression of a transgene in tissues from a transgenic non-human mammal can be assessed using techniques that include, but are not limited to, Northern blot analysis of tissue samples obtained from the animal, in situ hybridization analysis, and reverse-transcriptase PCR (RT-PCR). For example, a sample (e.g., tail tissue) can be collected from a transgenic mouse whose genome is suspected to contain a mutant hAPP transgene and an hAChE transgene. Nucleic acid molecules (e.g., DNA) can be purified from the sample using a commercially available kit (e.g., the Qiagen DNeasy Tissue Kit). The purified nucleic acid molecules can be used to genotype the transgenic mouse, e.g., by amplifying portions of the mutant hAPP and hAChE transgenes. Amplified reaction products can be separated by gel electrophoresis, and, based on the presence or absence of genotyping bands (e.g., 434 bp (hAPP) and/or 230 bp (hAChE)), the transgenic mouse can be classified as non-transgenic (e.g., no bands), mutant hAPP (e.g., only hAPP bands), hAChE (only hAChE bands), or doubly transgenic (e.g., both hAPP and hAChE bands). Segregation analysis of PCR products can be used to determine heterozygosity or homozygosity for each transgene.

Phenotype

A doubly transgenic non-human mammal of the invention also exhibits one or more useful phenotypes. A doubly transgenic mouse of the invention, for example, can exhibit an altered (e.g., increased) level of SDS-extractable amyloid beta. The level of SDS-extractable amyloid beta can be determined using a number of different methods known in the art. In one embodiment, the level of SDS-extractable amyloid beta in a mutant hAPP/hAChE transgenic mouse is determined by ELISA using amyloid beta capture antibodies (e.g., BNT77; Takeda, Osaka, Japan) and antibodies that recognize amyloid beta 1-40 (e.g., BA27; Takeda) or amyloid beta 1-42 (e.g., BC05; Takeda). An altered level of SDS-extractable amyloid beta can be from a 2-fold to a 20-fold or greater (i.e., 2-5-, 10-, 15-, 20-, 50-, or 100-fold or greater) increase or decrease compared to the level of SDS-extractable amyloid beta in a suitable control mouse (e.g., a non-transgenic mouse, a mutant hAPP singly transgenic mouse, or an hAChE singly transgenic mouse). Also, an altered level of SDS-extractable amyloid beta can range from 2 pmol/g to 20 pmol/g amyloid beta or greater at 6 months of age, or from 4 pmol/g to 90 pmol/g amyloid beta or greater at 9 months of age.

In addition, a doubly transgenic non-human mammal of the invention can exhibit an altered number of plaques in the brain when measured on or before 12 months of age (i.e., 1, 2, 5, 6, 7, 8, 9, 10, 11, or 12 months of age). The number of plaques in the brain can be determined using methods known in the art. In one embodiment, the number of plaques in the brain of a mutant hAPP/hAChE transgenic mouse can be determined by counting the number of thioflavin S-positive structures in histological brain sections per unit area. The number of plaques in doubly transgenic mice can be from 2-fold to 10-fold greater than the number of plaques in brain sections from a corresponding control mouse. An altered number of plaques can be from about 0.5 to about 1.0±0.25 SEM plaques/slide at 6 months of age or can be from about 3.5 to about 4.0±1.0 SEM plaques/slide at 9 months of age. The number of plaques per cm² can be calculated by multiplying the number of plaques per slide times 3.3.

Further, a doubly transgenic non-human mammal of the invention can exhibit early plaque formation. Early plaque formation can be determined using methods known in the art, including histological methods. In one embodiment, histological brain sections from mutant hAPP/hAChE transgenic mice euthanized at different ages (e.g., at 1, 2, 3, 4, 5, 6, 9, and 12 months) can be assessed for plaque formation as a function of time. Early plaque formation can be observed from about 5 months to about 6 months of age.

It is understood that the presence of a particular phenotype is assessed by comparing that phenotype to the corresponding phenotype exhibited by a control mouse lacking one or both transgenes. Thus, an increase in the number of plaques in the brain of a mutant hAPP/hAChE transgenic mouse at 6 months of age is, for example, relative to the number of plaques in the brain of a control mouse lacking one or both transgenes (e.g., containing a mutant hAPP transgene and not an hAChE transgene) at 6 months of age.

Screening

A doubly transgenic non-human mammal of the invention can be used as a test transgenic mammal to determine whether or not a candidate test substance can alter a particular phenotype exhibited by that mammal. Suitable candidate substances include chemical compounds, mixtures of chemical compounds, biological macromolecules (e.g., polypeptides), or biological materials such as extracts of bacteria, plants, fungi, and animals. Means are available for random and directed synthesis of a wide variety of organic compounds and biomolecules, including synthesis of randomized oligonucleotides and oligopeptides. Alternatively, libraries of natural compounds in the form of bacterial, fungal, plant, and animal extracts are available or can be produced. Natural or synthetically produced libraries and compounds can be modified using chemical or physical techniques known in the art, and such techniques can be used to produce combinatorial libraries. Known pharmacological agents may be subjected to directed or random chemical modifications, (e.g., acylation, alkylation, esterification, or amidification) to produce structural analogs. Generally a plurality of assay mixtures is run in parallel with different agent concentrations to obtain a differential response at the various concentrations.

A number of methods can be used as an end-point to determine whether a candidate test substance alters a particular phenotype exhibited by a double transgenic non-human mammal, and therefore may be considered as a potential therapeutic agent for neurodegenerative disorders. Such methods include without limitation, biochemical, histological, and behavioral assays. Biochemical assays, such as ELISA assays, are known in the art. For example, the level of SDS-extractable amyloid beta in a mutant hAPP/hAChE transgenic mouse treated with a test agent at a particular dose for a particular duration can be determined by ELISA, and compared to the level determined in a control mouse (e.g., an untreated mutant hAPP/hAChE transgenic mouse, a treated non-transgenic mouse, a treated or untreated transgenic mouse lacking one transgene, or a mutant hAPP/hAChE transgenic mouse treated with a different agent).

Histological assays can be used to determine whether on not an agent affects a particular phenotype exhibited by a transgenic non-human mammal. The average number of plaques in brain sections from a mutant hAPP/hAChE transgenic mouse can, for example, be determined and compared to the average number of plaques in brain sections from an appropriate control mouse.

Behavioral assays can be used to determine whether or not an agent affects a particular phenotype exhibited by a transgenic non-human mammal. Such assays include, without limitation, the Morris water maze and the radial arm water maze. For example, when employing the Morris water maze, a mutant hAPP/hAChE transgenic mouse is trained to swim to a submerged platform. The mutant hAPP/hAChE transgenic mouse can then be treated with an agent at a particular dose for a particular duration. Following treatment, the mean escape latency (i.e., time to find platform) is measured and compared to the mean escape latency of a control mouse.

When employing the radial arm water maze, a mutant hAPP/hAChE transgenic mouse is trained to swim a path to a submerged platform during a first day of testing. On a second day of testing, the platform can be moved to a different location, and the mutant hAPP/hAChE transgenic mouse can be trained to swim the new path to the submerged platform. The mutant hAPP/hAChE transgenic mouse can be treated with an agent at a particular dose for a particular duration at any time during the testing period. Following treatment, the ability of the transgenic mouse to remember a newly-learned path is measured and compared to that of a control mouse.

As can be seen in the following Examples, mice doubly transgenic for the Swedish mutation of APP as well as human ACHE, in accordance with the invention, were impaired in the radial arm water maze. Careful exclusion of animals with vision problems (rd homozygotes, red eyes) or which failed a cued platform test (thigmotaxis, lack of motivation or understanding of the task) enabled to detect statistically significant effects with relatively small numbers of animals. As compared to controls, the doubly transgenic animals were found to have more difficulty remembering the position of a platform within a day-long trial of 5 swims. Mice carrying the transgene for APPswe alone were also impaired in this task to a similar degree.

Interestingly, the simple hAChE expressers tended to perform better than controls in the radial arm water maze (p=0.07). These animals were previously reported to be impaired in the reference version of the Morris Water Maze [Beeri R, et al. Curr Biol (1995) 5(9):1063-71], as well as in the working memory test of social exploration [Cohen et al., Mol. Psych. 7:874-885 (2002)]. However, neither of these tests excluded mice with particularly poor performance and both showed considerable inter-animal variability. In the current test, hAChE mice with satisfactory vision and low anxiety were not impaired in working memory. In fact their learning curve was steeper (p<0.05) than those of the other tested genotypes (data not shown), compatible with the recently demonstrated correlation between brain AChE levels and working memory performance [Cohen et al. (2002) id ibid.].

By all present measures, the doubly transgenic APPswe/AChE animals exhibited increased AD like pathology as compared to APPswe mice, yet their average maze performance was no worse. Without being bound by theory, this finding might be related to a subtle effect of the hAChE genotype to protect memory as suggested by the improved performance of simple hAChE expressers. Alternatively, the possibility that doubly transgenic mice actually were impaired relative to the APP mice, but in a fashion that could only be determined by more difficult tasks, or with larger group numbers or at other developmental stages cannot be ruled out.

It has been shown that memory impairment in a working memory task and amyloid burden are weakly correlated in the APPswe mice, but are correlated to an impressive degree in doubly transgenic APPswe/hAChE mice. This high correlation together with the earlier onset of amyloid pathology means that studies with behavior as an endpoint can be conducted more efficiently with the transgenes of the invention.

The Examples show a strong correlation between amyloid deposition and cognitive impairment in mice doubly transgenic for human APPswe and human AChE. At nine months, these animals exhibited a striking increase in amyloid burden, measured by both histology and ELISA, as compared with controls or with singly transgenic APPswe mice. In a working memory version of a radial arm water maze, the same animals were impaired to a degree that was strongly predicted by the amyloid burden. These findings make the APPswe/hAChE mouse a desirable model to develop and test new compounds as potential AD therapeutics, particularly agents that are based on the amyloid hypothesis.

It is understood that when comparing phenotypes to assess the effects of a test agent, a statistically significant difference indicates that that particular test agent, test dosage, or test duration warrants further study. Typically, a difference in phenotypes is considered statistically significant a p≦0.05 with an appropriate parametric or non-parametric statistic, e.g., Chi-square test, Student's t-test, Mann-Whitney test, or F-test. In some embodiments, a difference in the number of plaques is statistically significant at p<0.01, p<0.005, or p<0.001. A statistically significant difference in, for example, the level of SDS-extractable amyloid beta in a test transgenic mouse treated with a test substance compared to the level in a control mouse indicates that (1) the substance alters the level of SDS-extractable amyloid beta, the number, density or burden of plaques and (2) the substance wan-ants further study as a candidate for treating neurodegenerative diseases such as AD.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Materials and Methods

All reagents were purchased from Sigma-Aldrich (St. Louis, Mo.) unless otherwise noted. Animals were handled under an IACUC-approved protocol, according to the NIH “Guide to Care and Use of Laboratory Animals”.

Generation of a Mutant hAPP/hAChE Transgenic Rodent

Male Tg2576 mice overexpressing a Swedish mutant human APP were mated with HpAChE female mice overexpressing human AChE. The Tg2576 mice are described in Hsiao, K., et al., Science 274:99-102 (1996). Additional information regarding mice overexpressing mutant human APP is found in U.S. Pat. No. 5,877,399. The HpAChE mice, and the constructs used to generate HpAChE mice, are described in U.S. Pat. No. 5,932,780. Gestation was about 21 days with an average litter size of 10 mice produced between 21 and 24 days from the initial mating. At postnatal day 21, the mice were weaned from their mother, and a tissue sample from the tip of the tail of each mouse was collected. DNA was purified from the tissue sample using the DNeasy Tissue Kit (Qiagen, Valencia, Calif.) according to the manufacturer's instructions. Once purified, the DNA was used in a PCR to genotype each mouse by amplifying a portion of the transgene of interest. Two separate reactions were performed to genotype the HAPP transgene as well as the hAChE transgene. The sequences of the primers were as follows:

| | |
|---|---|
| hAPP | |
| 5′-CTGACCACTCGACCAGGTTCTGGGT-3′ | (SEQ ID NO:1) |
| 5′-GTGGATAACCCCTCCCCCAGCCTAGACCA-3′ | (SEQ ID NO:2) |
| hAChE | |
| 5′-TCCTGGTGAACCACGAATGGC-3′ | (SEQ ID NO:3) |
| 5′-GGCTGATGAGAGACTCGTTGT-3′ | (SEQ ID NO:4) |
| θ-actin | |
| 5′-ACCCACACTGTGCCCATCTA-3′ | (SEQ ID NO:5) |
| 5′-CGGAACCGCTCATTGCC-3′ | (SEQ ID NO:6) |

Each PCR amplification cycle consisted of a denaturing step at 95° C. for 30 seconds, an annealing step for 15 seconds, and an elongating step. The annealing temperatures for the various primers were 59° C. for hAPP and θ-actin and 62° C. for hAChE. The elongation temperatures and times for the various primers were 72° C. for 30 seconds for hAPP and θ-actin, and 70° C. for 15 seconds for hAChE. Each reaction began with a single denaturing step at 95° C. for 15 minutes and ended with a single elongating step at 72° C. for 7 minutes. Once amplified, the reaction products were separated in a 1.6% agarose gel by electrophoresis. Based on the presence or absence of genotyping bands at 434 bp (hAPP) and/or 230 bp (hAChE), mice were classified as non-transgenic (no bands), Tg2576 singly transgenic (only hAPP bands), AChE singly transgenic (only hAChE bands), or doubly transgenic (both HAPP and hAChE bands). Progeny exhibited Mendelian inheritance of the two transgenes. Doubly transgenic mice were designated hAPP/hAChE transgenic mice.

Tissue Samples

Control and hAChE expresser mice were euthanized by ether overdose and their brains were rapidly removed (in some cases along with other tissues). Brain hemispheres were frozen on dry ice and stored at −80° C. pending use for histochemistry and ELISA measurements. In some cases, brains were microdissected into multiple regions before freezing, including parieto-frontal cortex, neostriatum, basal forebrain, hippocampus, and brainstem/cerebellum.

Selective Immunoprecipitation of hAChE

Microdissected brain samples were thawed and homogenized in 10 volumes of buffer (50 mM sodium phosphate, 1% Triton X-100, 0.1% BSA, 1 mM EDTA, pH 7.4). After centrifugation (10,000×g for 10 min), 50 μl aliquots of the supernatants were incubated for one hour at 37° C. with Pansorbin cells (Calbiochem, San Diego, Calif.) linked to a mouse monoclonal anti-human AChE antibody (HR2, 10⁻⁶M (Sussman, J. L. et al., Chemico-Biological Interactions 87:187-197 (1993)). The cells were then centrifuged and rinsed several times in buffer (50 mM sodium phosphate, 0.1% BSA). Finally, the pellets were resuspended in the original volume of the same buffer and 50 μl aliquots were assayed for bound human enzyme using the Ellman assay (Games, D. et al., Nature 373:523-527 (1995)) modified for a microplate reader. Assays were performed at 23° C. with 1 mM acetylthiocholine iodide as substrate in 0.1 M sodium phosphate, pH 8.0 with 0.1 mM ethopropazine to inhibit any BChE activity present. Samples not incubated with Pansorbin were also assayed to determine total activity in the tissue.

Sucrose Density Gradient Analysis

Supernatants of whole brain homogenates (diluted 1:10 in 50 mM sodium phosphate buffer, pH 7.4) of hAChE expressing and control mice were loaded onto 5-20% sucrose density gradients and spun at 150,000×g for 16 hours. Gradients were fractionated into 27 or 28 fractions and aligned with reference to a catalase marker. The fractions were incubated with HR2-linked Pansorbin cells (HR2 at 10⁻⁶M) for 1 hour at 37° C. The cells were centrifuged and rinsed before resuspension of the pellet and assay as above.

Histochemistry

Fresh frozen brain hemispheres were sectioned on a Leica cryostat in 15 groups of 3 adjacent slides at 12 μm thickness in 200 μm increments to prevent overestimation of plaque burden by redundant counting. Sections were fixed on the slides with 1% formalin for 2 hours at 4° C. prior to staining.

Beta-Amyloid

Thioflavin S (1% w/v, Sigma Chemical, St. Louis Mo.) was used as a general stain for amyloid deposits. After staining for 5 minutes in Mayer's hematoxylin (Sigma) to block nuclear staining, sections were stained with Thioflavin S for 5 minutes and differentiated in 70% ethanol for 5 minutes. Sections were rinsed in distilled water and coverslipped with glycerin jelly. Visualization was accomplished with an FITC filter on a Zeiss fluorescence microscope. For immunohistochemical confirmation, some slides were later de-coverslipped and treated with 100% formic acid (Sigma) for 5 minutes to remove the Thioflavin stain and make the amyloid accessible for antibody staining. The sections were then blocked with avidin-biotin (Vector, Burlingame, Calif.) and stained overnight at 4° C. with a biotinylated rabbit polyclonal antibody against Aβ 1-40 or 1-42 (Biosource, 1-40 at 1:400; 1-42 at 1:250, diluted in “TBS”, 0.9% NaCl, 100 mM Tris, pH 7.4). Slides were developed using the Elite ABC kit (Vector) with 3′,3′-diaminobenzidine (DAB, Sigma) as a chromagen to produce an insoluble brown deposit.

GFAP/CD11b

Avidin-biotin blocked sections were stained overnight at 4° C. with primary antibody, either polyclonal rabbit anti-mouse GFAβ (Zymed, South San Francisco, Calif. 1:300 in 5% normal goat serum (NGS), 0.1% bovine serum albumin (BSA), 0.1% Triton X-100 in TBS); or biotinylated monoclonal rat anti-mouse CD11b (Serotec, Raleigh, N.C., 1:500 in TBS). Glial fibrillary acidic protein (GFAP) antibody was followed by biotinylated goat anti-rabbit IgG (Vector, 1:500 in 5% NGS, 0.1% BSA, 0.1% Triton X-100 in TBS);

CD11b staining required no secondary antibody. Following development with the Elite ABC kit using DAB as chromagen, sections were counterstained with Thioflavin S as detailed above.

AChE

Sections were fixed on the slides with 1% formalin for 45 minutes at room temperature.

Primary antibody was a 1:500 dilution of HR2 mouse monoclonal anti-human ACHE antibody Staining was accomplished with the “Mouse-on-Mouse” protocol and reagents (Vector) to minimize background staining.

Plaque Staining and Quantitation

Sections were fixed on the slides with 1% formalin in 50 mM sodium phosphate buffer, pH 7.4 for two hours at 4° C. The remainder of the procedure was performed at room temperature. Following a rinse with 50 mM sodium phosphate buffer, slides were incubated five minutes each in Mayer's hematoxylin, deionized water, 1% Thioflavin S (made fresh every day), and 70% ethanol. Slides were rinsed in deionized water and mounted with glycerin jelly. Plaques were visualized with an FITC filter on a Zeiss fluorescence microscope (10× objective interfaced to a computer running NIH Image (version 1.57)). Plaque numbers and area were determined by hand tracing of the digitized images. Plaque burden was calculated by dividing total plaque area by total section area for each mouse sampled. Total section area was determined similarly using a dissection microscope and 3.2× objective. From each brain, 15 slides at 200 μm spacing were examined, and all plaques on the selected sections were analyzed.

Quantitative ELISA

ELISA measurements for Aβ were performed as described elsewhere Gravina, S. A. et al., id ibid.]. Brain hemispheres were homogenized first at 150 mg/ml in 2% w/v SDS (Sigma) in water with protease inhibitors (Roche, Indianapolis, Ind.) to recover soluble amyloid, then homogenized in 70% formic acid (Sigma) in water to obtain the insoluble amyloid fraction. Homogenates were spun at 100,000×g for 1 hour. Supernatants were diluted in buffer EC (0.02M sodium phosphate, pH 7.0, 0.2 mM EDTA, 0.4M NaCl, 0.2% bovine serum albumin, 0.05% CHAPS, 0.4% Block-Ace (DaiNippon Pharmaceutical, Osaka), 0.05% sodium azide) and transferred to ELISA plates coated with capture antibody BNT77 (Takeda, Osaka, recognizes Aβ residues 11-28). Secondary antibodies (Takeda) were HRP-conjugated BA27 (1-40) and BC05 (1-42). A peroxidase spectrophotometric detection system was used. Aβ was quantified by comparison with a standard curve of synthetic Aβ 1-40 and 1-42 (Bachem, Philadelphia, Pa.).

Statistics

Data were analyzed by non parametric statistics to avoid assumptions about normality of distribution. Group means were compared by Mann-Whitney test; frequency distributions were compared by Kolmogorov-Sminov test. P<0.05 was considered statistically significant.

Statistics

Maze behavior was analyzed by repeated measures ANOVA followed by Fisher's PLSD for post-hoc testing (StatView 4.5, Abacus Concepts, Berkeley Calif.). Anxiety and explorative behavior was analyzed using a factorial ANOVA and similar post-hoc tests.

Mouse Genotyping for Behavioral Tests

Tail tips were collected at the time of weaning and DNA was purified for PCR genotyping using described primers for APP and AChE as described above. In order to exclude animals with retinal degeneration (rd), the DNA for the associated gene (cGMP phosphodiesterase beta subunit) was also genotyped by restriction fragment length polymorphism (RFLP) as described [Pittler, S. J. et al. Proc. Natl. Acad. Sci. 88(19):8322-8326 (1991)]. For the rd analysis, DNA was amplified using primers:

W149:

5′ CAT CCC ACC TGA GCTCAC AGA AAG3′ (SEQ ID NO:7)

and W150:

5′ GCC TAC AAC AGA GGA GCT TCT AGC3′) (SEQ ID NO:8) at an annealing temperature of 55° C. for 30 cycles in a Perkin Elmer 2400 thermocycler. The PCR product was then digested with 2 units of Dde I (Promega, Madison Wis.) for two hours at 37° C. and run on an agarose gel.

Behavioral Testing

Exclusions: On grounds of suspected visual deficits, mice that had red eyes or were homozygous for rd were excluded from the testing. Additionally, mice that failed to find the platform in a visually cued test were excluded from analysis.

Radial Arm Water Maze: To test working memory, nine month old littermates from each genotype (control, hAChE, hAPPswe, and hAPPswe/hAChE) were swum in a blinded fashion in a 54.5″ diameter by 18 inch deep six-armed radial water maze (Little Tikes, Hudson, Ohio). The water in the maze was opacified with nontoxic white powdered tempera paint. The mice were given five one-minute trials per day on ten consecutive days to test their ability to remember the location of a submerged platform. Mice that failed to find the platform were placed on the platform for at least five seconds before being removed from the maze. The location of the platform changed every day, and was never in the same arm two days in a row. Mice were tracked using Ethovision Basic (Noldus, Amsterdam, Netherlands). Errors (entering the wrong arm or entering the correct arm without finding the platform) were tabulated for each mouse.

Cued platform test: After completing the trial series in the radial arm water maze, mice were tested in a cued version of a Morris Water Maze (the same maze with the plexiglass arm partitions removed). In this task the target platform stood above water and was marked with a checkered flag. Mice were excluded from further analysis if they failed to reach the platform in at least three of five one-minute trials.

Open field anxiety and exploration: To complete the testing battery, mice were analyzed in a standard open field anxiety and exploration task. They were placed in a 30 inch square clear plexiglass box with one-inch squares scored in the bottom. For five minutes, total squares crossed and the numbers of grooming events, urinations and defecations were noted with the use of a hand counter.

Tissue Preparation and Histology for Behaviorally Tested Animals

Following behavioral testing, mice were euthanized by ether overdose and their unperfused brains were cut into hemispheres, which were frozen on dry ice and stored at −80° C. for histological or ELISA analysis, as described above. Frozen brain hemispheres were cut in a Leica cryostat into 12-μm sections and adhered to Probe-On Plus slides (Fisher). Sections were cut in groups of three, spaced 200 μm apart to prevent overestimation of plaque burden. Slides were stored at −80° C. pending staining.

### Example 1

Early Onset of Amyloid Plaques in Brains with High AChE Expression

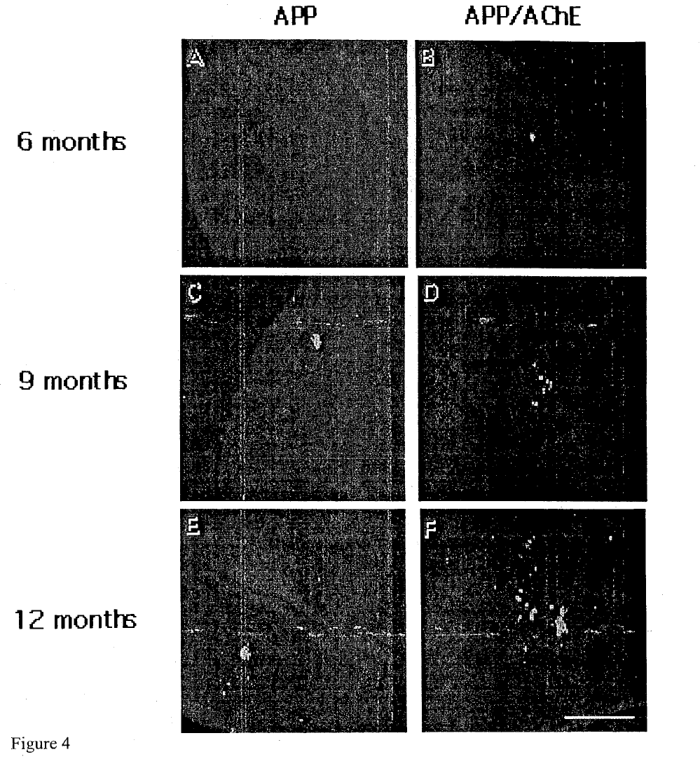

Amyloid plaques were initially examined by histochemistry with Thioflavin S. Over time, singly transgenic mice expressing human APP (hAPP) gradually developed Thioflavin S-positive amyloid plaques, primarily in cerebral cortex, hippocampus, and basal forebrain. Plaques were virtually absent at 6 months but were clearly evident at 9 months of age and became abundant (although slightly smaller on average than 9 months) at 12 months (FIG. 4, Table 1).

TABLE 1

| | | | | | | |
|---|---|---|---|---|---|---|
| | | | plaque size | Frequency | density | plaque burden |
| Age | Genotype | n | (sq. μm) | (plaques/section) | (plaques/sq cm) | (sq. μm/sq. cm) |

| | | | | | | | | | | |
|---|---|---|---|---|---|---|---|---|---|---|
| 6 months | APP | 8 | 520 ± | 130 | 0.08 ± | .03 | 0.56 ± | 0.21 | 120 ± | 65 |
| | APP/AChE | 10 | 480 ± | 54 | 0.73 ± | .09^{c} | 2.5 ± | 0.33^{a} | 1100 ± | 360^{a} |
| 9 months | APP | 8 | 1600 ± | 94 | 2.8 ± | .21 | 8.8 ± | 0.71 | 12000 ± | 2100 |
| | APP/AChE | 9 | 1100 ± | 60^{c} | 3.7 ± | .21^{b} | 12 ± | 0.72^{c} | 9600 ± | 1100 |
| 12 months | APP | 6 | 1300 ± | 86 | 19 ± | 2.3 | 66 ± | 7.9 | 48000 ± | 7700 |
| | APP/AChE | 6 | 1300 ± | 78 | 26 ± | 2.1^{b} | 96 ± | 12^{a} | 66000 ± | 7000 |

Samples were from transgenic littermates obtained by crossing hAPP-expressing Tg2576 mice with hAChE mice. Measurements were based on Thioflavin-S reactive amyloid deposits. Numbers represent means±SEM. Plaque burden was calculated by dividing total plaque area by total section area for each mouse sampled. Statistical significance versus age-matched controls was determined by Mann-Whitney test: ^{a}p<0.05 ^{b}p<0.01 ^{c}p<0.001.

It can be seen that plaques appeared even earlier in the doubly transgenic animals expressing both hAPP and hAChE. By 6 months, these mice showed small but clear-cut amyloid deposits at an average frequency of 2.4 per Cm², at least an order of magnitude more numerous than in singly transgenic hAPP mice of comparable age. Plaques in the doubly transgenic animals remained more numerous at both 9 and 12 months, but the rise in estimated amyloid burden was less consistent. At 9 months, for example, individual plaques were smaller than in singly transgenic HAPP expressers, but the total plaque area summed across all sections was equivalent in the two groups. At 12 months on the other hand, although attrition in the hAPP expressing lines left fewer mice for study, plaque size in the double transgenics was comparable to single transgenics, and total plaque area was increased, although not significantly.

### Example 2

Characterization of Amyloid Deposits in Doubly Transgenic Mice

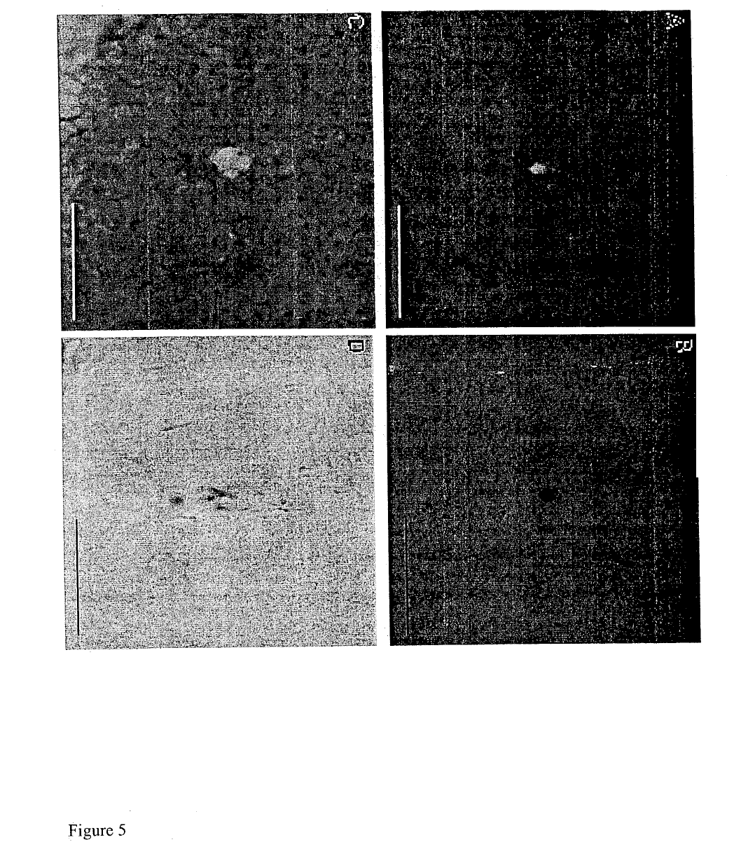

To determine whether the observed plaques were truly amyloid in nature, Thioflavin S staining with immunohistochemistry based on antibodies directed at Aβ 1-40 and 1-42 was followed (see Methods). Both antibodies co-localized with the Thioflavin S stain (FIG. 5), indicating that the plaques contained each of the two common isoforms of amyloid. Thioflavin also co-localized with a “pan-Aβ” antibody that recognizes C-terminally modified or truncated forms of amyloid in addition to the more common forms (data not shown). This pattern of co-localization appeared whenever plaques were detected, and was already established at six months of age in the doubly transgenic mice. Hence there is no doubt that the plaques represented genuine amyloid deposits in the brain tissue.

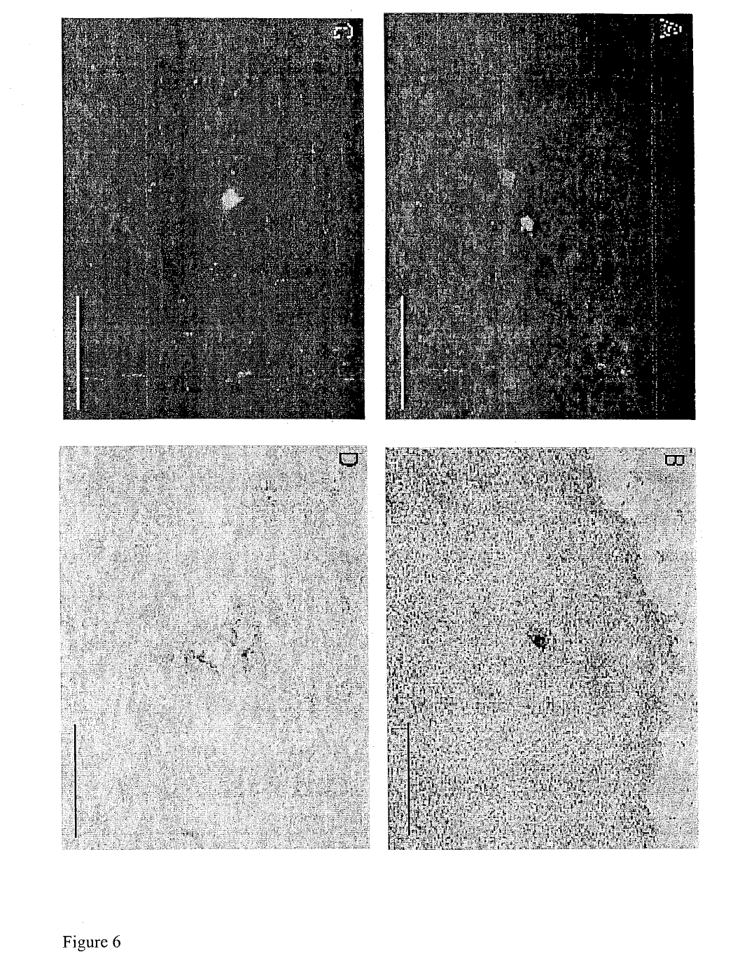

In parental Tg2576 mice, as in Alzheimer's Disease, maturing amyloid plaques are associated with a variety of markers that indicate activation of microglia and astrocytes. Consequently, the inventors elected to determine whether the early-onset plaques in doubly transgenic mice would meet this criterion for pathogenic relevance. For that purpose we immunostained brain sections from 6 month doubly transgenic mice for CD11b, a protein upregulated in activated microglia, and for GFAP, a marker of reactive astrocytes. The same sections were double labeled with Thioflavin S to identify candidate plaques. In all plaques, CD11b staining clustered closely around the Thioflavin-S positive core, while many surrounding GFAP-positive astrocytes projected processes inward (FIG. 6). Plaques identified in older doubly transgenic mice exhibited a similar structure, as did those in singly transgenic hAPP-expressing animals. By this criterion, all of these plaques can be considered classic, Alzheimer-type amyloid deposits.

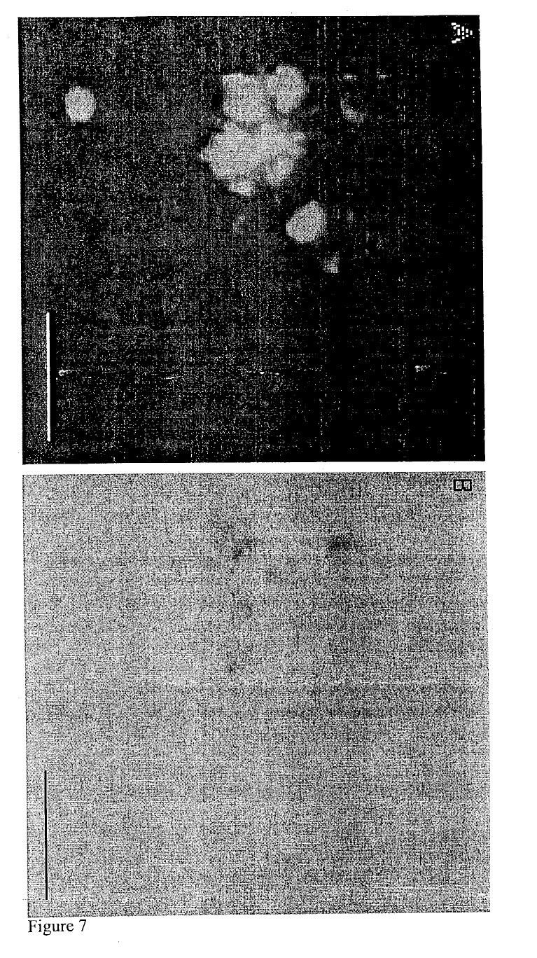

The doubly transgenic brains were also stained for human AChE to determine if the plaques contained AChE, as Alzheimer brains do. This question is central to the hypothesis of a direct interaction between AChE and amyloid beta. In fact, mature plaques consistently displayed AChE immunoreactivity (FIG. 7). AChE immunoreactivity also appeared, at lower intensity, in the earliest identified plaques at six months of age.

Amyloid Extractability in hAPP/hAChE Transgenic Mice

Brains were harvested from control (non-transgenic), Tg2576 (mutant hAPP) transgenic, hAChE transgenic (hAChE), and hAPP/hAChE doubly transgenic mice were treated as described in Methods. Each brain was treated individually according to the following procedure. Briefly, a brain hemisphere was weighed and then homogenized in 2% SDS (1 ml/150 g). The resulting homogenate was centrifuged at 100,000×g for 60 minutes. The supernatant containing the soluble amyloid fraction was decanted, divided into 50 μl aliquots, and saved for ELISA testing as the soluble SDS fractions. The insoluble amyloid fraction was released from the pellet by homogenizing in a volume of 70% formic acid equivalent to that of the original SDS extract. The resulting homogenate was centrifuged at 100,000×g for 60 minutes. The supernatant containing the released insoluble amyloid fraction was decanted, divided into 50 μl aliquots, and saved for ELISA testing as the insoluble formic acid samples.

Two soluble SDS aliquots and two insoluble formic acid aliquots each corresponding to the same brain hemisphere were transferred to an ELISA plate coated with a monoclonal capture antibody recognizing Aθ 11-28 (BNT77; Takeda). After 24 hours the ELISA plate was washed twice in PBS buffer and then treated with a cocktail of HRP-conjugated monoclonal antibodies recognizing human Aθ 1-40 and Aθ 1-42 (BA27 and BC05, respectively; 10-6 M dilution; Takeda). After 24 hours the ELISA plate was washed in PBS buffer and rinsed in PBS/Tween buffer. The HRP-conjugated antibodies were then developed using a peroxidase detection system, and the resulting signal was measured using a plate reader (Molecular Devices, Sunnyvale, Calif.) and Softmax software, version 2.35. Values were represented as the mean pmol/g±standard error (n=6 per group) and statistical significance was established by a Mann-Whitney nonparametric test.

The results showed that the soluble SDS fraction increased with time (see Table 2 at 6 and 9 months) in mutant hAPP/hAChE doubly transgenic mice compared to control, mutant hAPP, or hAChE littermates. These data demonstrate that mutant hAPP/hAChE doubly transgenic mice exhibit a statistically significant increase in the level of SDS-extractable amyloid beta in the brain when measured at 6 or more months of age.

TABLE 2
ELISA data from control, hAChE, mutant hAPP, and mutant hAPP/hAChE mice

| | | | Average pmol/g ± SEM | | | |
|---|---|---|---|---|---|---|
| Months | Genotype | n | SDS 1-40 | SDS 1-42 | FA 1-40 | FA 1-42 |
| 3 | control | 2 | 1.28 ± 0.080 | 0.919 ± 0.127 | 0 | 0 |
| | hAChE | 3 | 0.65 ± 0.091 | 0.91 ± 0.133 | 0 | 0 |
| | mutant hAPP | 7 | 19.0 ± 0.924 | 2.79 ± 0.162 | 0.054 ± 0.054 | 0.007 ± 0.007 |
| | hAPP/hAChE | 6 | 19.5 ± 1.14 | 2.89 ± 0.224 | 0 | 0 |
| 6 | hAChE | 5 | 1.07 ± 0.221 | 1.12 ± 0.040 | 0 | 0 |
| | mutant hAPP | 6 | 9.74 ± 4.12 | 1.97 ± 0.510 | 0.15 ± 0.145 | 0.07 ± 0.073 |
| | hAPP/hAChE | 7 | 11.9 ± 3.75 | 2.33 ± 0.377 | 0 | 0.04 ± 0.037 |
| 9 | hAChE | 1 | 1.11 | 1.17 | 0 | 0 |
| | mutant hAPP | 3 | 15.5 ± 1.59 | 2.59 ± 0.257 | 33.6 ± 11.8 | 28.3 ± 7.58 |
| | hAPP/hAChE | 6 | 41.8 ± 21.7 | 4.73 ± 0.756 | 72.9 ± 60.9 | 50.7 ± 47.1 |
| 12 | control | 4 | 1.16 ± 0.193 | 1.09 ± 0.109 | 0 | 0 |
| | hAChE | 5 | 1.14 ± 0.154 | 1.06 ± 0.123 | 0 | 0 |
| | mutant hAPP | 5 | 311 ± 119 | 46.5 ± 20.4 | 824 ± 273 | 364 ± 109 |
| | hAPP/hAChE | 2 | 273 ± 176 | 35.7 ± 17.5 | 2100 ± 1420 | 700 ± 473 |

hAChE = human acetylcholinesterase transgenic mouse
mutant hAPP = mutant human beta amyloid precursor protein transgenic mouse
hAPP/hAChE = double transgenic mouse
control = wild type mouse (no transgene)
SDS 1-40 = SDS extractable amyloid (soluble fraction) detected with the BA27 antibody
SDS 1-42 = SDS extractable amyloid (soluble fraction) detected with the BC05 antibody
FA 1-40 = Formic acid extractable amyloid (insoluble fraction) detected with the BA27 antibody
FA 1-42 = Formic acid extractable amyloid (insoluble fraction) detected with the BC05 antibody

### Example 3

Behavioral Testing in APP/AChE Mice

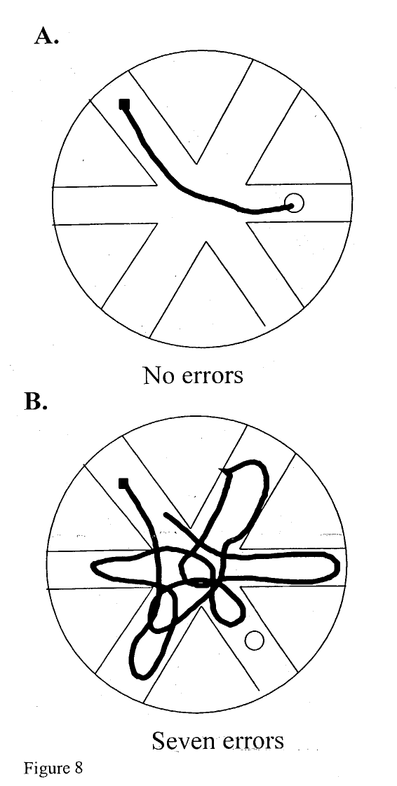

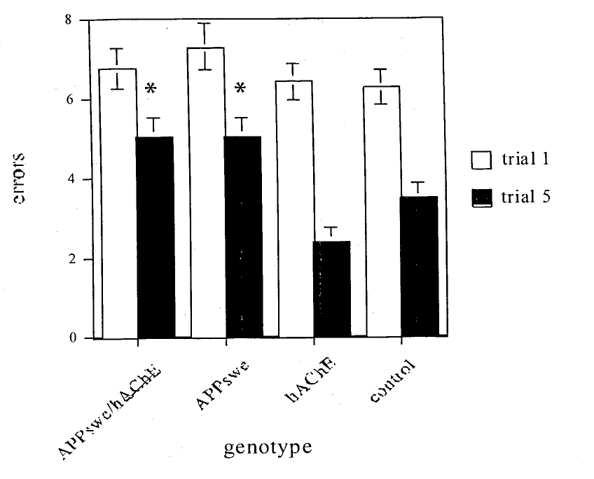

Littermate 9 month old mice from all relevant genotypes were tested in a radial arm water maze to assess working memory. FIG. 8 shows a schematic of the water maze as well as two typical swim paths. To avoid a potentially confounding effect of swim speed, errors rather than latency to reach the platform were recorded. Mice all started at the same level of skill, but by trial 5 of each day, the controls and ACHE expressers had cut their errors by over half whereas the APP and the doubly transgenics reduced them 25% at most (FIG. 9). Both groups that carry the APPswe gene were impaired in the task to approximately the same degree.

Exclusions based on failure to find the cued platform showed no genotype effect, with exactly three per genotype. The majority of excluded mice displayed profound thigmotaxis (wall hugging) during the cued test as in the radial maze.

To address the possibility that differences in anxiety levels or explorative behavior between genotypes accounted for the changes in maze performance, we assessed anxiety and explorative behavior (Table 4). A factorial ANOVA on each of these behaviors, however, showed no genotype effect.

TABLE 4

| | squares | grooming | | |
|---|---|---|---|---|
| genotype | crossed | behaviors | urinations | Defecations |
| doubly transgenic | 283 ± 28 | 4 ± .8 | .1 ± .1 | 2.1 ± .8 |
| App | 236 ± 27 | 4.5 ± 1.0 | 0 | 1 ± .4 |
| Ache | 199 ± 22 | 4 ± .8 | 0 | 1.1 ± .5 |
| Control | 291 ± 21 | 4.5 ± .9 | .1 ± .1 | 1.8 ± .7 |

Following completion of the radial arm water maze and cued water maze tasks, mice were tested to determine level of anxiety and propensity to explore new situations. Numbers represent means±SEM. A factorial ANOVA for each behavior was nonsignificant

### Example 4

Histology on Behaviorally Tested Mice

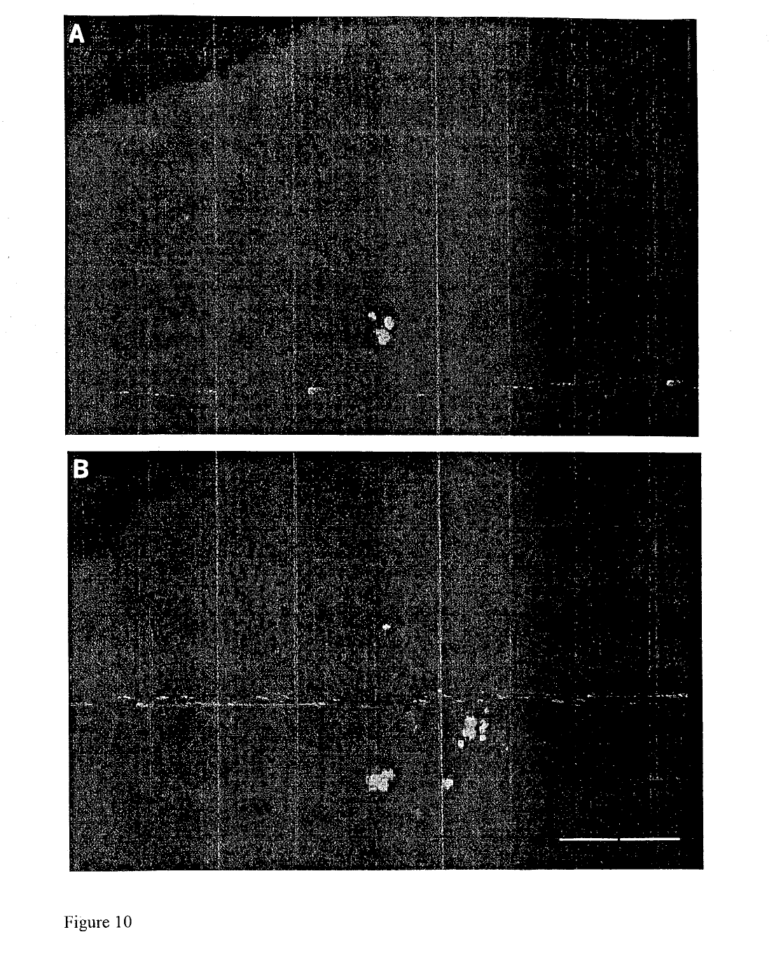

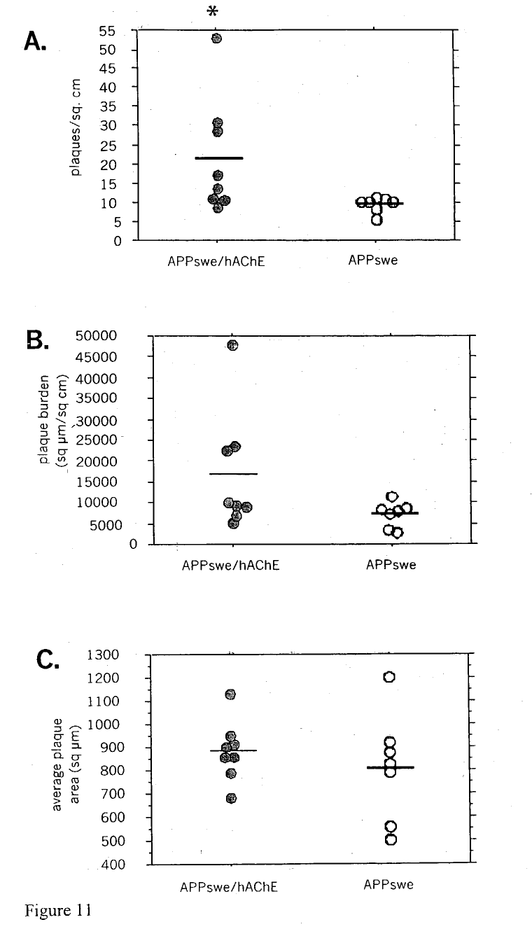

After behavioral testing, animals were examined histologically to assess the number of thioflavin-S reactive plaques and to determine plaque burden. Neither the control animals nor the hAChE expresser displayed any plaques or exhibited a measurable amount of insoluble amyloid. Therefore, the inventors focused on comparing the APPswe expresser with the APPswe/AChE mouse. A representative thioflavin S stained section from each genotype is shown in FIG. 10. Characteristically, the doubly transgenic mice had at least twice as many thioflavin-S reactive plaques in their cortex as did the APP expressers. Quantitation of the plaque density (plaques/sq. cm), plaque burden, and average individual plaque area is shown in FIG. 11. The doubly transgenic animals had an increased plaque density and plaque burden, but equivalent plaque sizes, indicating that the increase in plaque burden in the APP/AChE animals is fully explained by the increase in plaque number.

### Example 5

ELISA for Amyloid in Behaviorally Tested Mice

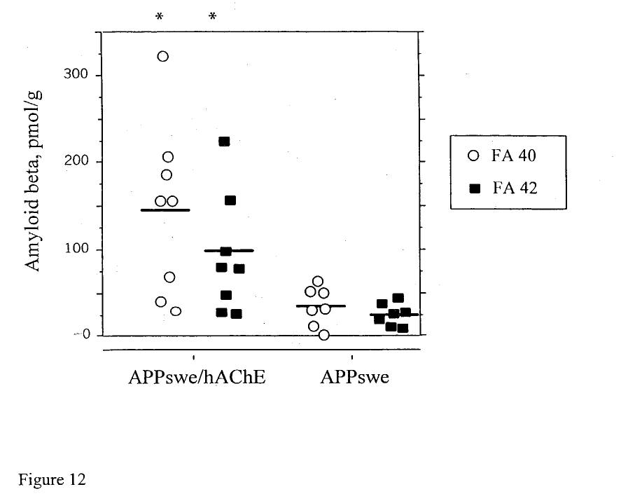

An ELISA to quantitate insoluble Aβ 1-40 and 1-42 was performed on one brain hemisphere from each behaviorally characterized mouse (FIG. 12). The ELISA data demonstrate a two fold increase in amyloid 40 and 42 in the doubly transgenic animals as compared to the APP animals, as in the above experiments.

### Example 6

Correlations Between ELISA, Amyloid Plagues, and Maze Performance

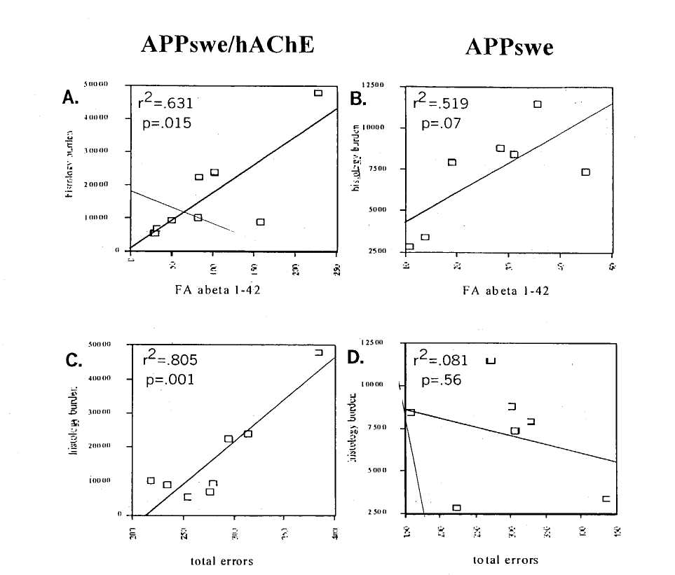

Mouse by mouse correlations were analyzed to gain insight into the finding that, despite a significantly increased amyloid burden, the AChE/APP mice do no worse on the spatial memory task than the APP mice. Plaque burden correlated positively with insoluble amyloid beta 1-42 in both genotypes (FIG. 13A,B). Since plaque burden was estimated from multiple individual observations per brain, this measure tends to be less variable than a single determination of amyloid by ELISA. Therefore, greatest stress was on correlating histological plaque burden for a given mouse with its total number of errors in the radial arm maze. Plaque burden correlated positively (r^{2=0.81}, n=8, p=0.001) with maze errors in the APPswe/AChE group (C), but not the APPswe only group (D). Owing to higher variability in the ELISA data, the correlations between errors made and biochemical estimates of amyloid burden were much poorer (r=0.560, n=8, p=0.15 for Aβ 1-42). The inventors' conclusion was that histological amyloid burden was a much better predictor of errors in spatial memory tasks in the doubly transgenic mice.

1. A transgenic rodent (or organism) having a genome comprising a mutant APP transgene and an AChE transgene, which transgenic rodent displays an altered deposition of amyloid beta in the brain, compared to a corresponding transgenic rodent whose genome comprises a mutant APP transgene and lacks an AChE transgene and progeny thereof.

2. The transgenic rodent according to claim 1, wherein said rodent is a mouse.

3. The transgenic rodent according to any one of claims 1 and claim 2, wherein the AChE transgene encodes a wild-type AChE polypeptide.

4. The transgenic rodent according to claim 3, wherein said wild-type AChE polypeptide is a full-length human AChE polypeptide.

5. The transgenic rodent according to claim 4, wherein the mutant APP transgene encodes a human APP mutated polypeptide.

6. The transgenic rodent according to claim 5, wherein said APP mutated polypeptide is selected from the group consisting of APP695, APP751, app563, app714 and APP770, and has a mutation selected from the group consisting of K670M, N671L, V717I, V717G, or V717F.

7. The transgenic rodent according to claim 6, wherein the genome of said transgenic rodent comprises a K670MIN671L mutant APP transgene and full-length human AChE polypeptide transgene.

8. The transgenic rodent according to claim 1, which exhibits deposition of plaques in its brain, the number of thioflavin-S reactive plaques being statistically significantly increased compared to the number of plaques in a corresponding transgenic rodent whose genome has a mutant APP transgene and lacks an AChE transgene.

9. The transgenic rodent according to claim 8, wherein the statistically significant increase occurs earlier in age compared to a corresponding transgenic rodent whose genome comprises a mutant APP transgene and lacks an AChE transgene.

10. The transgenic rodent according to claim 9, wherein the statistically significant increase is observed at an age of from about 6 to about 9 months of age.

11. The transgenic rodent according to any one of claims 9 and 10, wherein said rodent is a mouse.

12. The transgenic mouse according to claim 11, wherein the level of SDS-extractable amyloid beta in the brain of said mouse is altered compared to a corresponding transgenic mouse whose genome comprises a mutant APP transgene and lacks an AChE transgene and progeny thereof.

13. The transgenic mouse according to claim 12, wherein the level of SDS-extractable amyloid beta is elevated compared to a corresponding transgenic mouse whose genome comprises a mutant APP transgene and lacks an AChE transgene and progeny thereof.

14. A method of screening for a candidate substance for the treatment of a neurodegenerative disorder, which screening method comprises the steps of:
(a) providing a test transgenic rodent whose genome comprises a mutant APP transgene and an AChE transgene;
(b) administering said test substance to said transgenic rodent under suitable conditions; and
(c) determining the effect of the test substance on an end-point indication, wherein said effect is indicative of the therapeutic effect of said test substance on said neurodegenerative disorder; and optionally
(d) comparing said end-point indication with that of a corresponding control transgenic rodent not treated with said test substance.

15. The screening method for a candidate substance for the treatment of a neurodegenerative disorder, according to claim 14, which screening method comprises the steps of:
(a) providing a test transgenic rodent whose genome comprises a K670M/N671L mutant APP transgene and full-length human AChE polypeptide transgene;
(b) administering said test substance to said transgenic rodent under suitable conditions; and
(c) determining the effect of the test substance on an end-point indication, wherein said effect is indicative of the potential therapeutic effect of said test substance on said neurodegenerative disorder; and optionally
(d) comparing said end-point indication with that of a corresponding control transgenic rodent not treated with said test substance

16. The screening method according to claim 0.15, wherein said test transgenic rodent has a statistically significantly elevated level of SDS-extractable amyloid beta and increased number of thioflavin-S reactive plaques compared to a corresponding transgenic rodent whose genome comprises a mutant APP transgene and lacks an AChE transgene.

17. The screening method according to claim 14, wherein said end-point is the level of SDS-extractable amyloid beta obtained from a brain tissue of said transgenic rodent.

18. The screening method according to claim 17, wherein a decrease in the level of SDS-extractable amyloid beta obtained from said test transgenic rodent brain tissue compared to the level of SDS-extractable amyloid beta in said control transgenic rodent not treated with said test substance, is indicative of therapeutic effect of said substance on said neurodegenerative disorder.

19. The screening method according to claim 18, wherein said end-point is any one of the number, density and burden of thioflavin-S reactive plaques in the brain of said transgenic rodent as determined histologically.

20. The screening method according to claim 19, wherein a decrease in the number of thioflavin-S reactive plaques in the brain of said test transgenic rodent compared to the number of said plaques in the brain of said control transgenic rodent not treated with said test substance, is indicative of a therapeutic effect of said substance on said neurodegenerative disorder.

21. The screening method according to claim 14, wherein said end-point is a cognitive capacity as evaluated by a behavioral assay.

22. The screening method according to claim 21, wherein said capacity is working memory.

23. The screening method according to claim 21, wherein said behavioral assay is any one of Morris water maze, the radial arm water maze and open field anxiety and exploration test.

24. The screening method according to claim 21, wherein improvement of said cognitive capacity in said test transgenic rodent compared to the same cognitive capacity of said control transgenic rodent not treated with said test substance, is indicative of a therapeutic effect of said substance on a neurodegenerative disorder.

25. The method according to claim 14, wherein said neurodegenerative disorder is Alzheimer's disease.

26. The screening method according to claim 25, wherein said test substance is selected from the group consisting of: protein based, carbohydrate based, lipid based, nucleic acid based, natural organic based, synthetically derived organic based, metals and antibody based substances.

27. The screening method according to claim 26, wherein said protein, nucleic acid, chemical or antibody based substance is a product of a combinatorial library.

28. A method of preparing a therapeutic composition for the treatment of a neurodegenerative disorder, which method comprises the steps of:
(a) identifying a substance having a therapeutic effect on a neurodegenerative disorder by the screening method according to claim 14; and
(b) admixing said inhibitor with a pharmaceutically acceptable carrier.
